(19) United States
(12) Patent Application Publication
(10) Pub. No.: US 2022/0285039 A1
(43) Pub. Date: Sep. 8, 2022

(54) TELEHEALTH SYSTEMS

(71) Applicant: AndorHealth, LLC, Orlando, FL (US)

(72) Inventors: Raj Toleti, Orlando, FL (US); Balavignesh Thirumalainambi, Ridgewood, NJ (US); Pritesh Patel, Orlando, FL (US)

(73) Assignee: AndorHealth, LLC, Orlando, FL (US)

(21) Appl. No.: 17/685,088

(22) Filed: Mar. 2, 2022

Related U.S. Application Data
(60) Provisional application No. 63/156,258, filed on Mar. 3, 2021.

(51) Int. Cl. G16H 80/00; G16H 10/60; G06Q 30/04

(52) U.S. Cl. CPC G16H 80/00; G16H 10/60; G06Q 30/04

(57) ABSTRACT

A telehealth system can include a telehealth conferencing module configured to allow a provider to communicate with a patient, and a health information module configured to provide patient medical record information to the provider to the telehealth conferencing module. The system can include a billing module operatively associated with the telehealth conferencing module and the health information module to receive service information relating to a telehealth visit and provide a billing output.

### CROSS-REFERENCE TO RELATED APPLICATIONS

This application claims priority to and the benefit of U.S. Provisional Application No. 63/156,258, filed Mar. 3, 2021, the entire contents of which are herein incorporated by reference in their entirety.

### FIELD

This disclosure relates to telehealth systems.

### BACKGROUND

The American Medical Association and the Center for Medicare and Medicaid Services (CMS) have overhauled the coding required to bill for a patient office visit in a medical practice. These changes went into effect on Jan. 1, 2021. Healthcare providers and medical practices are finding it challenging to implement the changes to their current billing and coding practices based on these changes. This affects their reimbursement and increases the administrative time required to process the billing.

In the current workflow the medical practitioner will have to identify the patient in the EMR/EHR and will have to click through multiple screens to identify all the relevant information needed to successfully complete the patient evaluation. The medical practitioner will have to document the number and complexity of the problems addressed, amount and/or complexity of data to be reviewed and risk of complications and/or morbidity and mortality of patient management in different screens within the EHR and the billing staff will have to switch between these screens to bill for the encounter appropriately. The medical practitioner will have to switch between the EMR/EHR and the virtual health platform that they use. This process is not only time consuming, but also reduces the time the practitioner spends with the patient and is frustrating for the practitioner.

Following this the medical billing team will review the documentation in the EMR/EHR that was performed by the medical practitioner and will identify any gaps that may exist in the documentation. The billing team will then reach out to the medical practitioner to fix any issues in the documentation. This process is manual, inefficient, and can lead to billing inaccuracies and missed charges. This can also result in human errors and can be resource intensive, especially for smaller medical practices. There is a need for a solution that reduces this burden and makes the process more efficient, intuitive, which will in turn give the provider to focus on the patient and the administrative staff in order to reduce the overhead costs.

Such conventional methods and systems have generally been considered satisfactory for their intended purpose. However, there is still a need in the art for improved virtual health systems. The present disclosure provides a solution for this need.

### SUMMARY

A telehealth system can include a telehealth conferencing module configured to allow a provider to communicate with a patient, and a health information module configured to provide patient medical record information to the provider to the telehealth conferencing module. The system can include a billing module operatively associated with the telehealth conferencing module and the health information module to receive service information relating to a telehealth visit and provide a billing output.

In certain embodiments, the billing module can be configured to aggregate the service information. The billing output can include a billing file having the service information. For example, the billing file can be a PDF file.

The billing module can be configured to determine one or more billing strategies for the provider. In certain embodiments, the billing output can include the one or more billing strategies.

The one or more billing strategies can include a plurality of billing strategies. The billing module can be configured to select a proposed billing strategy from the plurality of billing strategies.

The billing module can be configured to output the proposed billing strategy. The billing module can be configured to determine one or more tasks based on substantive information in the service information and to look up a task value for each task from a task database.

The billing module can be configured to determine a billing time based on one or more of a manual time entry and/or a telehealth visit duration in the service information. The billing module can be configured to provide a graphical user interface configured to receive a manually input time entry from a provider.

In certain embodiments, the billing module can be configured to select the proposed billing strategy based on a comparison of total billing time value versus total task value based on aggregated task values. The billing module can be configured to provide the proposed billing strategy in a billing file. In certain embodiments, the billing module can be configured to automatically formulate and output details related to the patient encounter using the proposed billing strategy.

The telehealth conferencing module can include a videoconference pane and a health information pane. The health information module can be configured to output the patient medical record information to the health information pane.

The health information pane can include an interface for a provider to indicate whether the patient medical record information is reviewed. In certain embodiments, the billing module can be configured to confirm one or more billing tasks based on provider input to the health information pane.

In accordance with at least one aspect of this disclosure, a non-transitory computer readable medium comprising computer executable instructions configured to cause a computer to perform a method. The method can include connecting a healthcare provider to a patient via a telehealth conferencing module, receiving patient medical record information from an electronic medical record database, displaying the patient medical record information in a health information pane, receiving service information from the telehealth conferencing module, receiving pre-teleconference time entry input and/or post-teleconference time entry input from the healthcare provider, determining a billing output based on the service information, and outputting a billing file. Determining the billing output can include selecting a billing strategy based on a comparison of total billing time value versus total task value. The method can include any other suitable method(s) and/or portion(s) thereof.

In accordance with at least one aspect of this disclosure, a telehealth system can include a health information module configured to provide patient medical record information to a telecommunication platform during a telehealth visit between a patient and a medical provider, and a billing module operatively associated with the telecommunication platform and the health information module to receive service information relating to a telehealth visit and provide a billing output. The system can be or include any suitable portions of any suitable embodiments of a system disclosed herein, e.g., as described above.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a schematic diagram of an embodiment of a telehealth system in accordance with this disclosure;

FIG. 2 is a schematic diagram of an embodiment of the telehealth system of FIG. 1, shown in use with a teleconferencing pane and a chat pane, and also showing an embodiment of a bot in accordance with this disclosure receiving instructions and providing health record information relating to lab results associated with the patient;

FIG. 2A shows a close up view of the chat pane of FIG. 2;

FIG. 3 is a schematic diagram of an embodiment of the telehealth system of FIG. 1, shown in use with a teleconferencing pane and a chat pane, and also showing an embodiment of a bot in accordance with this disclosure receiving instructions and providing health record information relating to medications associated with the patient;

FIG. 3A shows a close up view of the chat pane of FIG. 3;

FIG. 4 is a schematic diagram of an embodiment of the telehealth system of FIG. 1, shown in use with a teleconferencing pane and a chat pane, and also showing an embodiment of a bot in accordance with this disclosure receiving instructions and providing visit duration information and/or input fields for the provider to input data such as pre-visit time spent;

FIG. 4A shows a close up view of the chat pane of FIG. 4;

FIG. 5 is a schematic diagram of an embodiment of the telehealth system of FIG. 1, showing a chat pane, and also showing an embodiment of bot in accordance with this disclosure receiving instructions and providing visit duration information and/or input fields for the provider to input data such as pre-visit time spent and post-visit time spent;

FIG. 5A shows a close up view of the chat pane of FIG. 5;

FIG. 6 is a schematic diagram of an embodiment of the telehealth system of FIG. 1, shown in use with a teleconferencing pane and a chat pane, and also showing an embodiment of a bot in accordance with this disclosure receiving instructions and providing an embodiment of billing output;

FIG. 6A shows a close up view of the chat pane of FIG. 6;

FIG. 7 shows a schematic of an embodiment of a provider dashboard of the system of FIG. 1, wherein the provider can access information relating to each patient including clinical information and/or billing information, for example, showing a clinical information pane at the forefront configured to allow the provider to mark that the lab results have been reviewed, e.g., to trigger a billing function;

FIG. 7A shows a close up view of the clinical information pane of FIG. 7;

FIG. 8 is a flow diagram of an embodiment of a billing automation method;

FIG. 8A is a flow diagram of an embodiment of a pre-visit portion of the method of FIG. 8;

FIG. 8B is a flow diagram of an embodiment of a during-visit portion of the method of FIG. 8; and

FIG. 8C is a flow diagram of an embodiment of post-visit portion of the method of FIG. 8.

### DETAILED DESCRIPTION

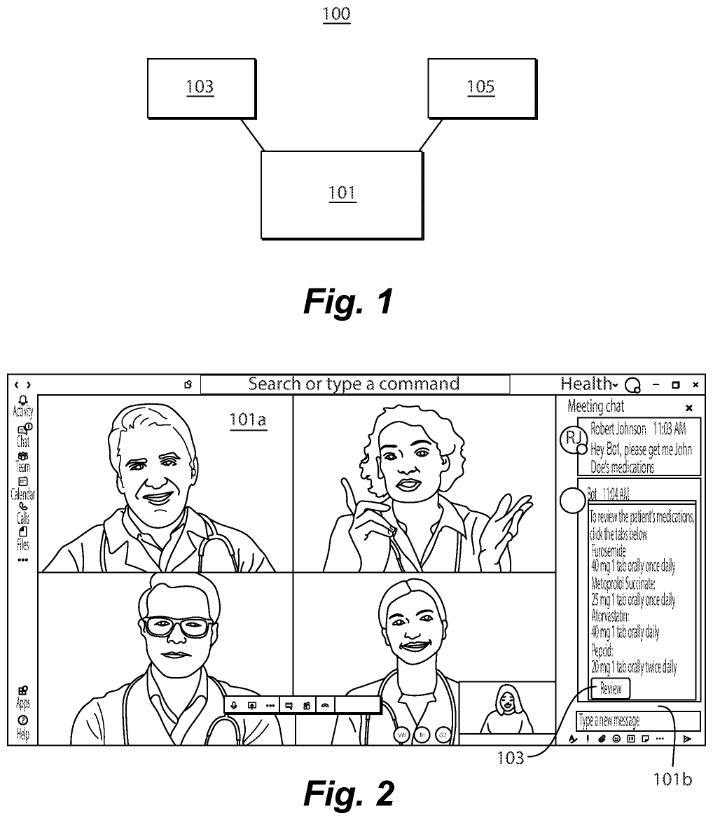

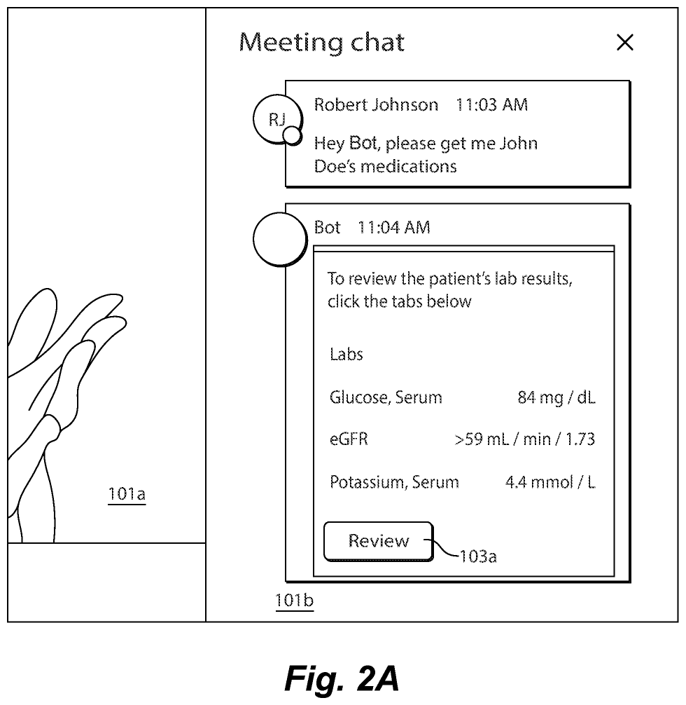

Reference will now be made to the drawings wherein like reference numerals identify similar structural features or aspects of the subject disclosure. For purposes of explanation and illustration, and not limitation, an illustrative view of an embodiment of a telehealth system in accordance with the disclosure is shown in FIG. 1 and is designated generally by reference character 100. Other embodiments and/or aspects of this disclosure are shown in FIGS. 2-8C. Certain embodiments described herein can be used to provide artificial intelligence enabled billing assistance for virtual health visits.

Referring to FIG. 1, a telehealth system 100 can include a telehealth conferencing module 101 configured to allow a medical provider to communicate with a patient. The telehealth conferencing module 101 can be configured to provide one or more of audio, visual, and/or textual communication between a medical provider and a patient. In certain embodiments, the telehealth conferencing module can include Microsoft Teams, Zoom, and/or any other suitable platform. In certain embodiments, the telehealth conferencing module 100 can be a proprietary platform.

The system 100 can include a health information module 103 configured to provide patient medical record information (e.g., clinical data) to the provider to the telehealth conferencing module 101 (e.g., within a chat pane of the telehealth conferencing module 101). For example, the health information module 103 can be operatively connected to an electronic medical records (EMR) database and can be configured to pull the health information associated with a patient to present it to the provider, either automatically or with manual control by the medical provider.

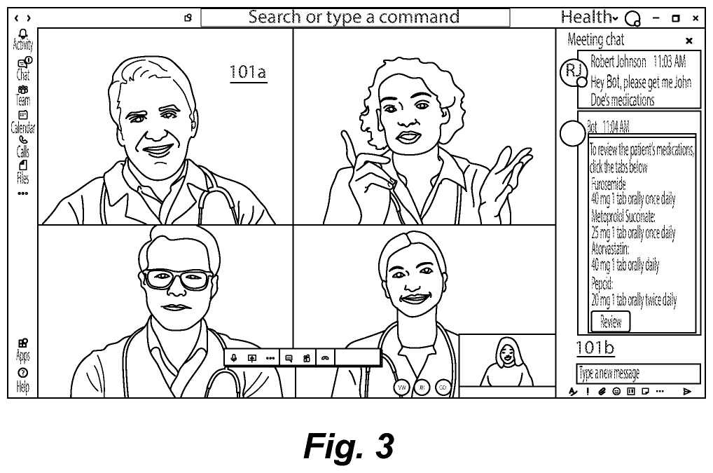

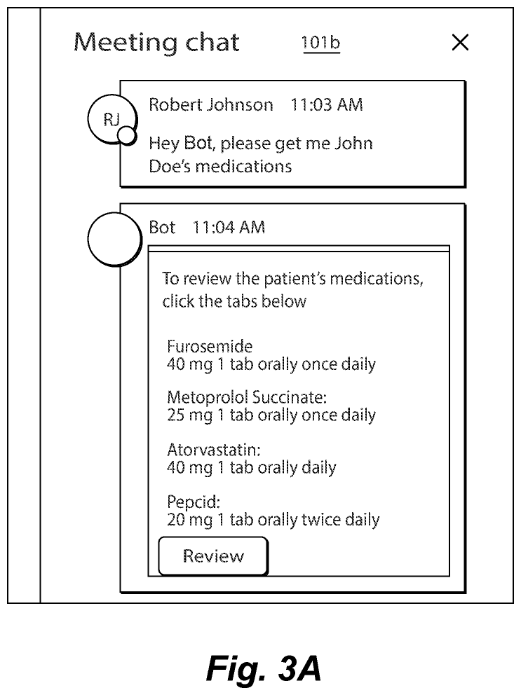

In certain embodiments, the health information module 103 can include artificial intelligence (AI) configured to review an EMR and pull relevant clinical data associated with a topic (e.g., associated with a topic input by the provider), for example. Any suitable intelligence and/or machine learning is contemplated herein. For example, as shown in FIGS. 2 and 2A, and FIGS. 3 and 3A, the health information module 103 can be configured to receive a command to retrieve certain information from an EMR (e.g., labs as shown in FIGS. 2-2A, and medications as shown in FIGS. 3-3B), and to retrieve and display related information accordingly (e.g., as shown).

The system can include a billing module 105 operatively associated with the telehealth conferencing module 101 and the health information module 103 to receive service information (e.g., duration-of-visit, substantive topics of discussion, medical provider input) relating to a telehealth visit and provide a billing output.

In certain embodiments the health information module 103 and the billing module 105 can form and/or be part of a bot module 107 that is configured to overlay and/or otherwise interact with the telehealth conferencing module 101. For example, the bot module 101 can be configured to output text and/or function items (e.g., buttons) to the chat pane and/or receive textual commands from the provider via the chat pane of the telehealth conferencing module 101. Any other suitable overlay and/or input/output pathway between the telehealth conferencing module 101 and the modules 103, 105, 107 is contemplated herein.

In certain embodiments, the system 100 may not include the telehealth conferencing module 101, and the system 100 can comprise the bot 107 configured to operate with any suitable conferencing platform. Any suitable configuration is contemplated herein.

In certain embodiments, the billing module 105 can be configured to aggregate the service information (e.g., to create a list of tasks completed and visit duration). The billing output can include a billing file, for example, having the service information. For example, the billing file can be a PDF file. Any other suitable file type(s) are contemplated herein. In certain embodiments, the billing output can be text presented in a pane of the telehealth conferencing platform.

In certain embodiments, the billing module 105 can be configured to determine one or more billing strategies for the medical provider. In certain embodiments, the billing output can include the one or more billing strategies (e.g., showing time-based billing vs. task-based billing results).

The one or more billing strategies can include a plurality of billing strategies (e.g., at least time-based billing and task-based billing). In certain embodiments, the billing module 105 can be configured to select a proposed billing strategy from the plurality of billing strategies (e.g., whichever strategy yields the highest amount of compensation to the provider). For example, task based billing or hourly based billing can result in different total values, and the higher of the two can be selected.

The billing module 105 can be configured to output the proposed billing strategy (e.g., in the billing file or otherwise). In certain embodiments, the billing module 105 can be configured to determine one or more tasks based on substantive information (e.g., what topics/clinical data was addressed in the meeting) in the service information and to look up a task value for each task from a task database (e.g., Medicare and/or insurer task billing rates). For example, the billing module 105 can include suitable AI configured to determine one or more of what topics were discussed, for how long, what clinical information was reviewed or discussed, and/or any what other services were provided to the patient during, before, and/or after the telehealth visit.

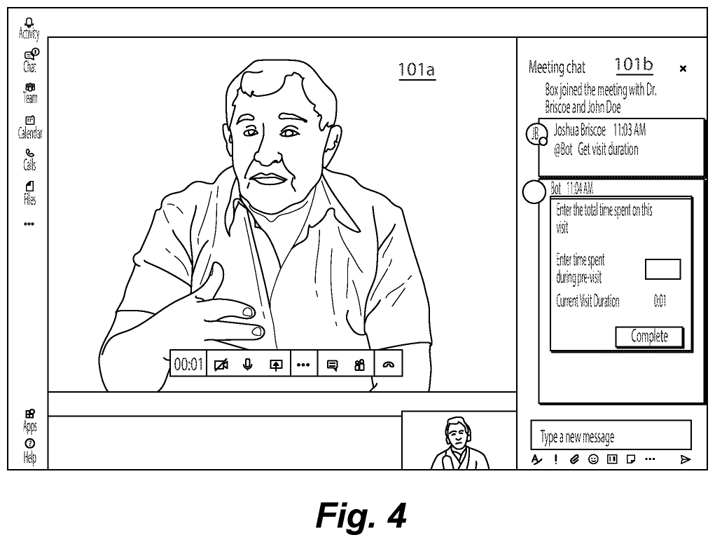

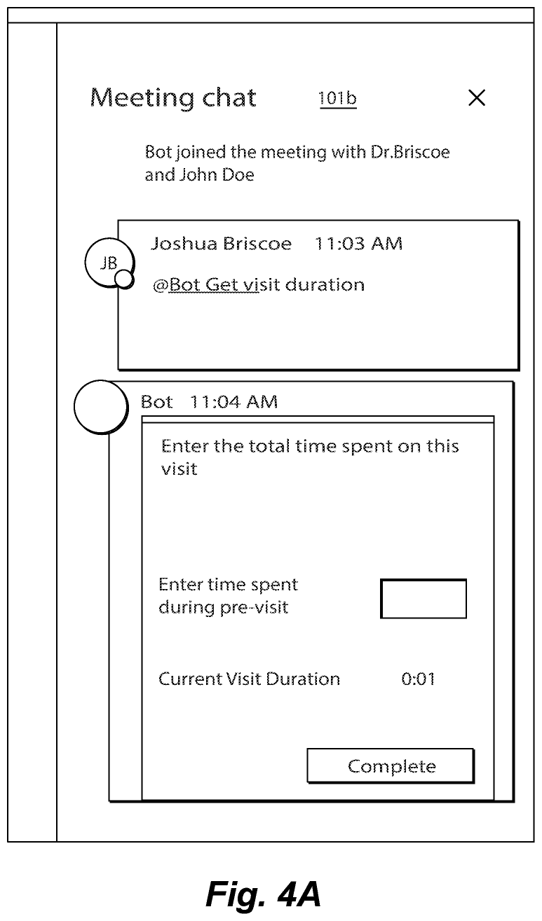

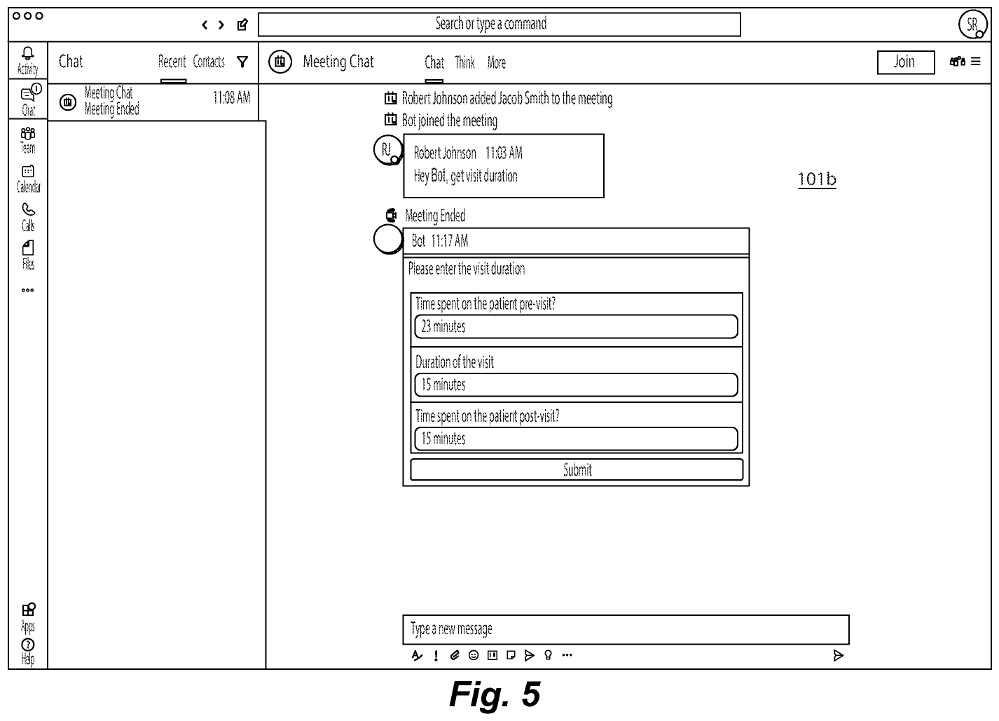

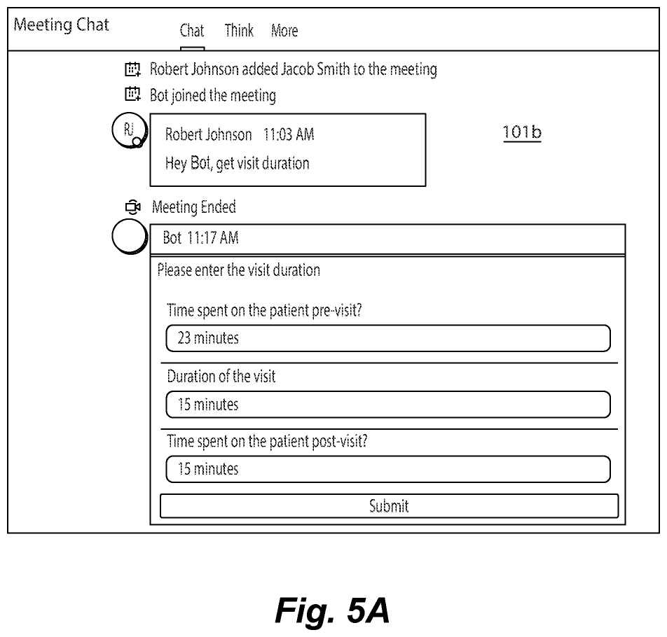

The billing module 105 can be configured to determine a billing time based on one or more of a manual time entry (e.g., time before or after the telehealth visit) and/or a telehealth visit duration (e.g., which can be automatically determined or provided by the teleconference platform) in the service information. For example, in certain embodiments, the billing module 105 can be configured to provide a graphical user interface (GUI) configured to receive a manually input time entry from a provider, e.g., as shown in FIGS. 4, 4A, 5, and 5A).

In certain embodiments, the billing module 105 can be configured to select the proposed billing strategy based on a comparison of total billing time value versus total task value based on aggregated task values. The billing module 105 can be configured to provide the proposed billing strategy in a billing file. In certain embodiments, the billing module 105 can be configured to automatically formulate and output details relating to the telehealth visit (e.g., an invoice) using the proposed billing strategy.

In certain embodiments, referring to FIGS. 2-6A, the telehealth conferencing module 101 can include a videoconference pane 101a and a health information pane 101b. In certain embodiments, the health information pane 101b can be a chat pane as shown (e.g., already existing or integrated with the teleconferencing module 101. The health information module 103 can be configured to output the patient medical record information to the health information pane 101b (e.g., as shown).

The health information pane 101b can include an interface 103a (e.g., a GUI button) for a provider to indicate whether the patient medical record information is reviewed. Utilizing this interface 103a can provide information (e.g., timestamp, textual information about the subject matter) to the billing module 105 to use in creating the billing output. For example, in certain embodiments, the billing module 105 can be configured to confirm one or more billing tasks based on provider input to the health information pane 101b (e.g., such that the billing module 105 concludes that a related task is complete and can be billed for).

In certain embodiments, the bot 107 can be configured to join the meeting as a participant through teleconferencing module 101. The bot 107 can be interacted with as a virtual assistant via the health information pane 101b (e.g., the chat pane) such that the bot 107 can take information and also post information to the health information pane 101b.

FIG. 2 is a schematic diagram of an embodiment of the telehealth system of FIG. 1, shown in use with a teleconferencing pane and a chat pane, and also showing an embodiment of a bot 107 in accordance with this disclosure receiving instructions and providing health record information relating to lab results associated with the patient. FIG. 2A shows a close up view of the chat pane of FIG. 2.

FIG. 3 is a schematic diagram of an embodiment of the telehealth system of FIG. 1, shown in use with a teleconferencing pane and a chat pane, and also showing an embodiment of a bot 107 in accordance with this disclosure receiving instructions and providing health record information relating to medications associated with the patient. FIG. 3A shows a close up view of the chat pane of FIG. 3.

FIG. 4 is a schematic diagram of an embodiment of the telehealth system of FIG. 1, shown in use with a teleconferencing pane and a chat pane, and also showing an embodiment of a bot 107 in accordance with this disclosure receiving instructions and providing visit duration information and/or input fields for the provider to input data such as pre-visit time spent. FIG. 4A shows a close up view of the chat pane of FIG. 4.

FIG. 5 is a schematic diagram of an embodiment of the telehealth system of FIG. 1, showing a chat pane, and also showing an embodiment of bot 107 in accordance with this disclosure receiving instructions and providing visit duration information and/or input fields for the provider to input data such as pre-visit time spent and post-visit time spent. FIG. 5A shows a close up view of the chat pane of FIG. 5.

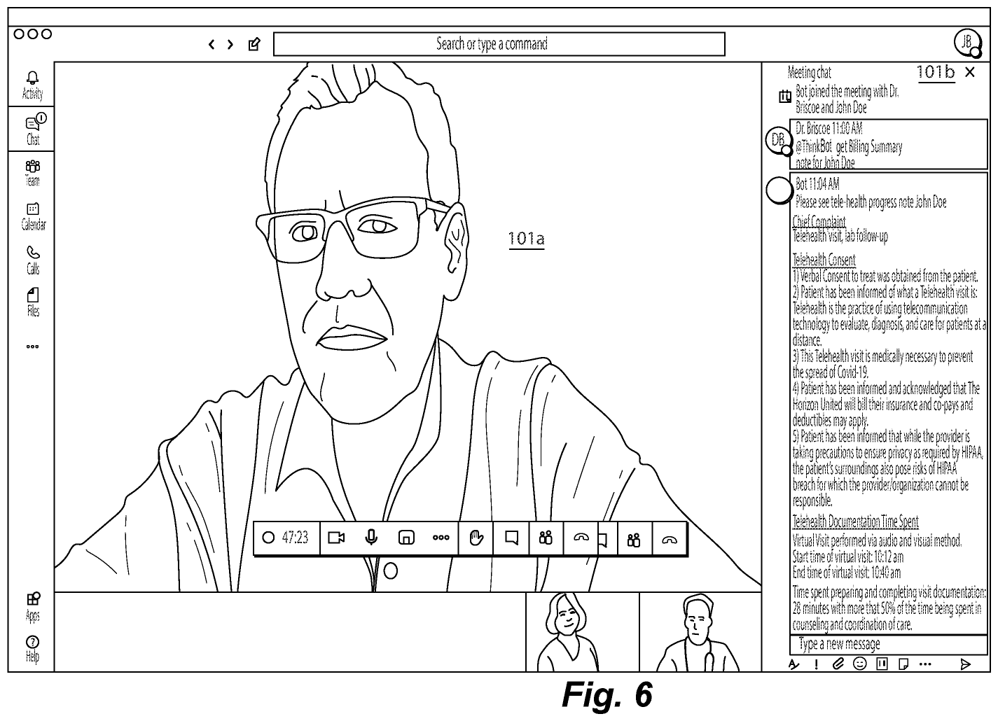

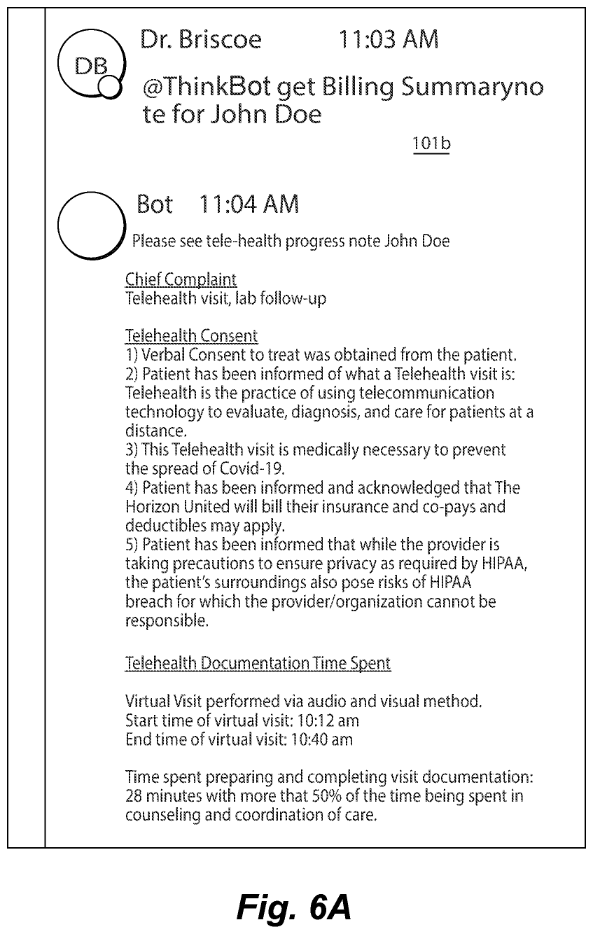

FIG. 6 is a schematic diagram of an embodiment of the telehealth system of FIG. 1, shown in use with a teleconferencing pane and a chat pane, and also showing an embodiment of a bot 107 in accordance with this disclosure receiving instructions and providing an embodiment of billing output. FIG. 6A shows a close up view of the chat pane of FIG. 6.

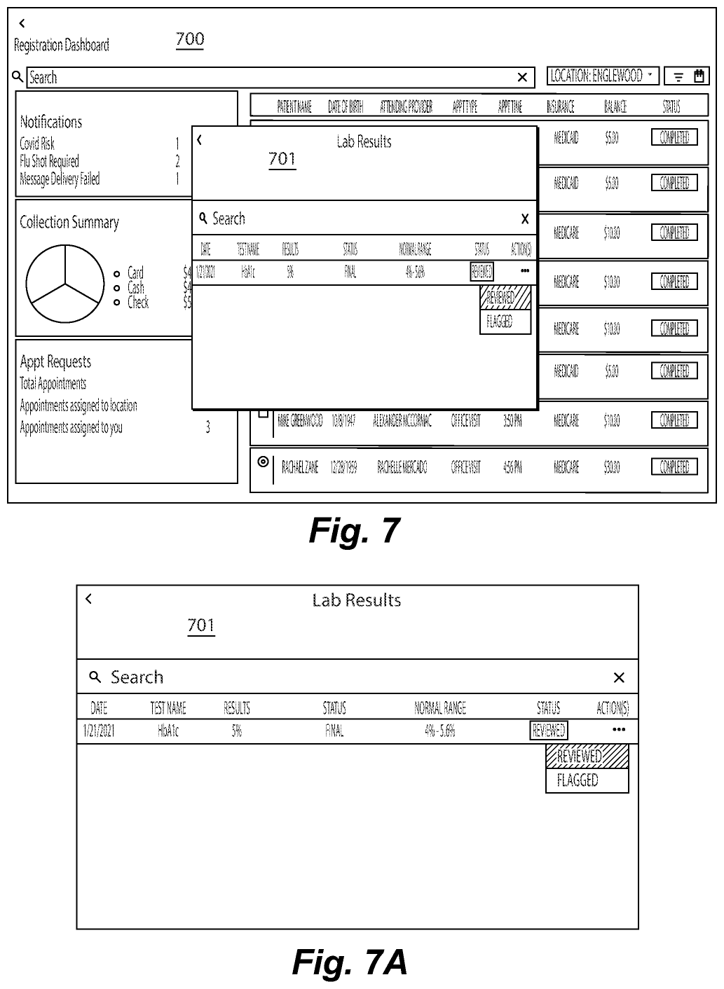

FIG. 7 shows a schematic of an embodiment of a provider dashboard 700 of the system 100 of FIG. 1. In certain embodiments, the system 100 and/or dashboard 700 can be provided as software-as-a-service (SAAS). Any other suitable form (e.g., downloadable software) or combination thereof is contemplated herein. The provider can access information relating to each patient including clinical information and/or billing information, for example. FIG. 7 shows a clinical information pane at the forefront configured to allow the provider to mark that the clinical information (e.g., lab results) has been reviewed, e.g., to trigger a billing function. FIG. 7A shows a close up view of the clinical information pane of FIG. 7.

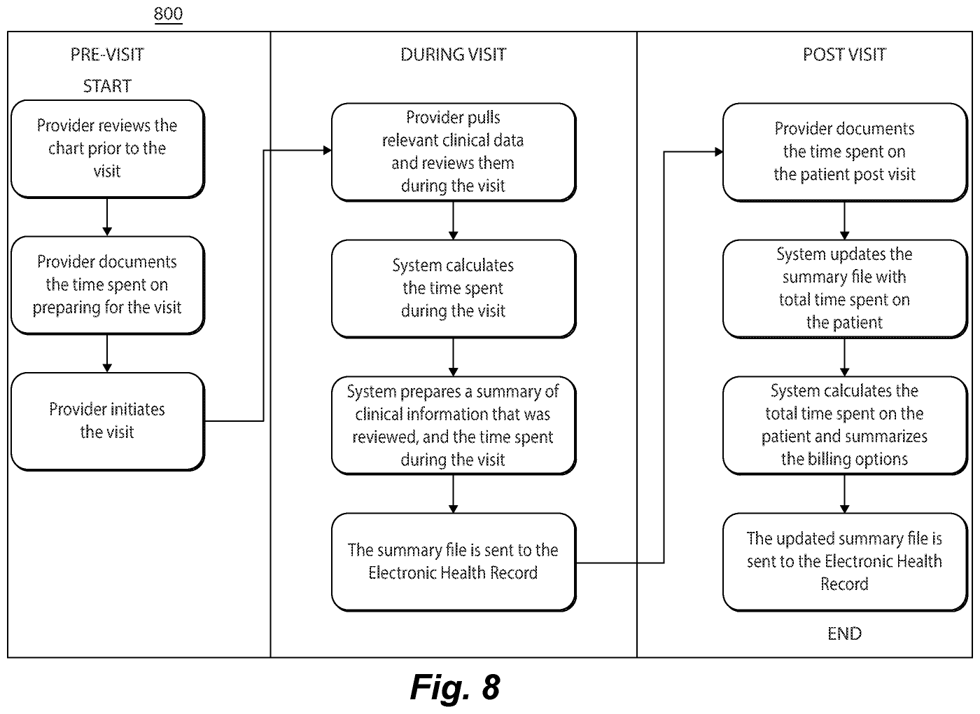

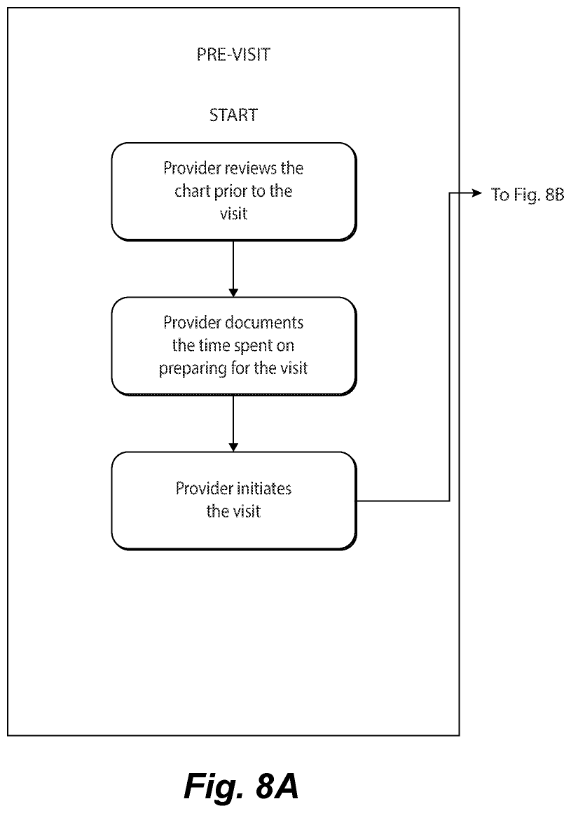

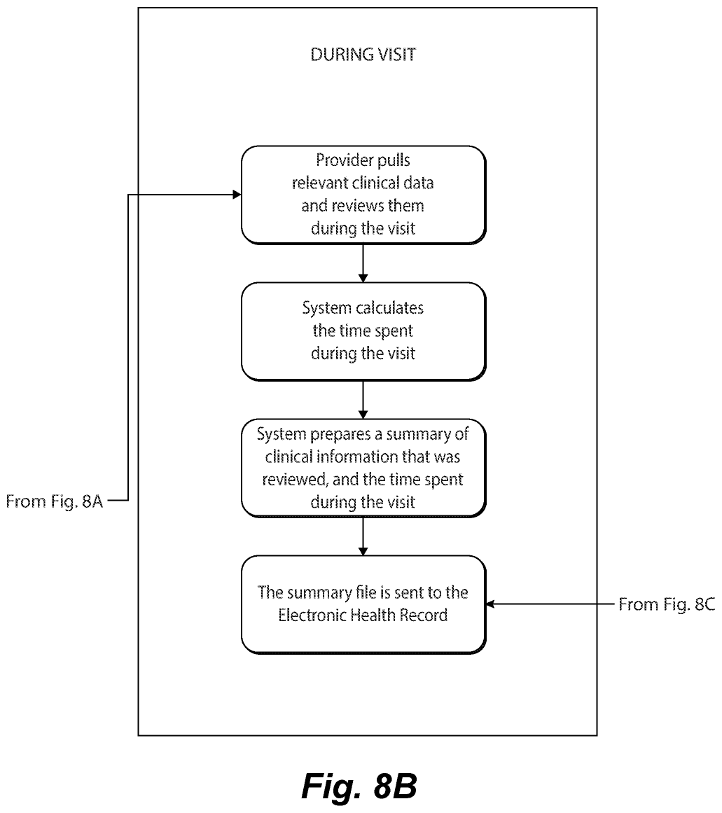

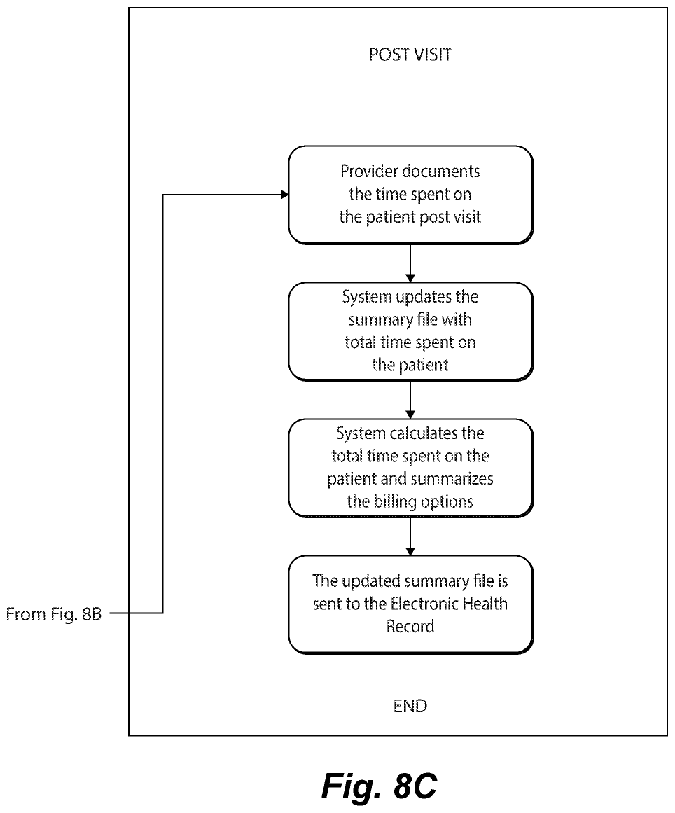

FIG. 8 is a flow diagram of an embodiment of a billing automation method in accordance with this disclosure. FIG. 8A is a flow diagram of an embodiment of a pre-visit portion of the method of FIG. 8. For example, the provider can review a chart before conducting a telehealth visit and can document and/or input into the dashboard 700 the time spent preparing for the visit before initiating the visit.

FIG. 8B is a flow diagram of an embodiment of a during-visit portion of the method of FIG. 8. During the telehealth visit, the provider can pull relevant clinical data using the health information module 103 (e.g., as shown in FIGS. 2-3A). The billing module 105 can calculate the time spent during the visit (e.g., by initiating a counter, recording timestamps for each action, and/or by receiving visit duration at one or more points from the telehealth conferencing module 101. The billing module can prepare a summary of clinical information that was reviewed and the time spent during the visit. In certain embodiments, a summary file can be sent to the electronic health record.

FIG. 8C is a flow diagram of an embodiment of post-visit portion of the method of FIG. 8. The provider can document the time spent on the patient post visit (e.g., inputting time entries into the billing module 105), and/or update the summary file with the total time spent on the patient. The billing module 105 can then take all relevant service information (e.g., time entries, tasks completed, etc.) and calculate billing options for the provider to select from. A final billing output and/or summary file can be sent to the EMR.

In accordance with at least one aspect of this disclosure, a non-transitory computer readable medium comprising computer executable instructions configured to cause a computer to perform a method. The method can include connecting a healthcare provider to a patient via a telehealth conferencing module, receiving patient medical record information from an electronic medical record database, displaying the patient medical record information in a health information pane, receiving service information from the telehealth conferencing module, receiving pre-teleconference time entry input and/or post-teleconference time entry input from the healthcare provider, determining a billing output based on the service information, and outputting a billing file. Determining the billing output can include selecting a billing strategy based on a comparison of total billing time value versus total task value. The method can include any other suitable method(s) and/or portion(s) thereof.

In accordance with at least one aspect of this disclosure, a telehealth system can include a health information module configured to provide patient medical record information to a telecommunication platform during a telehealth visit between a patient and a medical provider, and a billing module operatively associated with the telecommunication platform and the health information module to receive service information relating to a telehealth visit and provide a billing output. The system can be or include any suitable portions of any suitable embodiments of a system disclosed herein, e.g., as described above.

Embodiments of a system can include a health information module (e.g., a health bot) and a billing module operatively associated therewith. Embodiments can include a health information module that pulls info from a health file (e.g., an electronic health record) into a telehealth conferencing module (e.g., Microsoft Teams) during a telehealth visit to provide substantive information to be reviewed and discussed during the telehealth visit. Embodiments can include a billing module configured to additionally aggregate information that the health information module already pulls, to optionally take a manual time entry from a provider pre-visit, to optionally calculate and output an automated time entry for the telehealth visit on the conferencing platform (or this could be done manually), and to optionally take a manual time entry from after the visit. The billing module can be configured to link the time entry information (whether manual or automated) with the substantive information in the visit, and to output a data summary file linking the time entries and the substantive information for use in billing.

Embodiments of a graphical user interface (GUI) can include a “Review” button (e.g., in the same displayed card by the health information module) associated with certain substantive information (e.g., lab results or other medical information shown in the figures in a bot provided data card in the side bar of a video conferencing platform), which can be clicked by the provider to allow the provider credit and to put this substantive information in the data summary file. In certain embodiments, when the “Review” button is clicked, the billing module can record a timestamp for reviewing what substantive information was associated with the “Review” button.

In certain embodiments, the billing module can be configured to determine the most efficient billing strategy for the provider based on a comparison of billed time value versus billed task value (e.g., based on fixed Medicare task rates). The billing module can be configured to provide a recommendation of billing strategy in the output data summary file (e.g., to select which strategy yields the highest rate for the provider, thereby incentivizing the provider to provide the best service to the patient without being budget strategy focused).

The system can include any suitable hardware module(s) and/or software module(s) and can be configured to perform any suitable function (e.g., as disclosed herein or otherwise).

In accordance with certain embodiments, a virtual health solution can be used by a medical practice to conduct a patient office visit. The information related to a patient's office visit can be stored in the medical practice's electronic medical record or electronic health record (EMR/EHR) system. Using Artificial Intelligence (AI), relevant clinical information from the EMR/EHR can be extracted and displayed in the virtual health solution. With the relevant clinical information, the system can display options for the medical provider to conduct a review necessary to accurately bill for the office visit. This recommendation can be based on changes that were introduced for office visit coding by American Medical Association (AMA) and Center for Medicare and Medicaid Services (CMS). The system can capture the time spent by the medical practitioner during, prior to and after the visit in relation to the patient. Certain embodiments can advise the provider on the ideal type of billing, e.g., based on medically appropriate history and/or examination and Medical Decision Making (MDM) or total time on the date of the encounter, e.g., whichever is most beneficial.

Embodiments relate to the healthcare field, more specifically to medical practices and their billing process improvement. Embodiments include the process of pulling information from the HER (also referred to as EMR) and displaying it in the virtual health solution in a manner that is easily readable. This process can be pre-loaded or on-demand depending on the preference of the medical provider. The solution also allows the provider to document the number and complexity of the problems addressed, amount and/or complexity of data to be reviewed and risk of complications and/or morbidity and mortality of patient management using the virtual health solution.

Virtual health or telehealth can be defined as the delivery and facilitation of health and health-related services including medical care, provider and patient education, health information services, and self-care via telecommunications and digital communication technologies. The clinical information can be pulled into the virtual health visit by typing in commands or using voice commands to retrieve the relevant medical information. The system can retrieve information such as digital images, laboratory results, EKG results, radiology results, etc and display it in the virtual health solution. The information that can be retrieved from the EHR for a visit can include name, date of birth, provider, insurance, appointment date, start time, end time, for total time, previous discharge/visit summaries or other relevant clinical documentation, chief complaint/number of diagnoses, visit type, history of present illness as it pertains to the visit, allergies, medications, family history, social history, past medical and surgical history, labs, radiology, EKG tracing/interpretation, review of systems, physical exam, assessments, and plans. Any suitable information is contemplated herein.

Embodiments of a system can enable the medical practitioner to obtain the following information useful for the billing process, how the visit was performed via audio and visual, time spent completing visit documentation, verbal consent, whether patient informed telecommunication was used for evaluation, informed insurance can be billed, and informed information can be protected to ensure privacy as required by HIPAA.

In certain cases, a medical practice or the provider has options to code a particular office visit in two different ways. They can either code the visit based on medical decision making or based on the amount of time spent with the patient. If it is based on medical decision making, then it can be comprised of three elements:

(1) Problems Addressed: The number of problems that the practitioner addressed with the patient.

(2) Items Reviewed: Lab results, Imaging results and other information that was reviewed.

(3) Risk: The level of risk presented to the patient.

If the coding is based on time then the provider can include any time spent on the patient that day, including the time spent prior to, during and after the visit. Any other suitable way to bill for patient services is contemplated herein (e.g., hybrid billing).

FIG. 8 shows an example of a billing automation workflow in accordance with this disclosure. The bot 107 can display the data retrieved from the EHR in the chat portion and can be displayed as cards (e.g., as shown in FIGS. 2 and 2A). These cards can contain information such as digital images, laboratory results (e.g., as shown in FIGS. 2 and 2A), medication (e.g., as shown in FIGS. 3 and 3A), EKG results, radiology results. This can be triggered by typing commands such as “Get lab results” or “Get EKG results”. Any suitable command is contemplated herein.

The healthcare provider may be presented with a button at the bottom of each displayed item and can be able to click “Reviewed” (e.g., as shown in FIGS. 2 and 2A). Clicking “Reviewed” can record the time and date the information was reviewed by the provider. For EKG/Radiology studies, there can be an additional button to document that the medical practitioner also interpreted the results, e.g., in order to gain additional points towards the clinical decision making process. This information can be stored in the virtual health database and can later be transmitted to EHR database using HL7 and API standards.

Embodiments can automatically calculate the duration of the visit (e.g., as shown in FIGS. 4 and 4A). The bot 107 can calculate the starting point and the ending point of the visit and can calculate the total duration. In addition to this, the provider can also be able to enter the amount of time spent prior to the visit and after the visit on the patient. For example, by typing “Get visit duration”, the provider may be presented with a form that can allow them to enter the additional time spent on reviewing notes, results, correspondence with other clinicians, reports pre and post-visit (e.g., as shown in FIGS. 5 and 5A).

At the end of the visit, the bot 107 can also collect all the data points related to the “Reviewed” process and can prepare a summary (e.g., as shown in FIGS. 6 and 6A), which can be in the PDF format for example. This summary can include the number and complexity of the problems addressed, amount and/or complexity of data to be reviewed and risk of complications and/or morbidity and mortality of patient management as notes from the medical practitioner. Any suitable information for the summary is contemplated herein.

In certain embodiments, the provider can be provided with an option of either dictating these notes or typing them in to the system. The summary file can then be transmitted back to the EHR and can be stored under the patient's file, for example. In certain embodiments, a billing team can retrieve the summary file and can utilize it to identify all the components that were completed.

In certain embodiments, the billing module can present the provider with a billing summary file, e.g., as shown. In certain embodiments, the billing module can be configured to append the billing summary file to the health record (which can be accessed by a billing department, for example).

In certain embodiments, the system 100 can be configured such that a post visit review screen can allow the provider to manually select/deselect what was reviewed during the visit for accuracy (e.g., as shown in FIG. 7). Any other suitable functions are contemplated herein. Certain embodiments provide a method to allow the medical providers review relevant clinical data in a convenient way, where they are pulling relevant clinical data based on a command are clicking a “reviewed” button under the relevant clinical data within the virtual health visit environment. Certain embodiments provide a method to summarize all the actions taken by the provider during the virtual health visit and create a pdf file with a proprietary data structure.

Certain embodiments provide a method to calculate the total amount of time spent by the clinician on a particular patient, pre, post and during the visit. The actual visit time can be calculated automatically by the virtual health platform by recording the start time and the end time, the pre and post visit time will be calculated by allowing the medical provider to enter the time spent on the patient within the virtual health visit environment. Certain embodiments provide a method to identify which method of billing is more beneficial for the medical practice and presenting that in the pdf file with the proprietary data structure.

Any suitable embodiment and/or portions thereof (e.g., modules 101, 103, 105, bot 107) disclosed above can be embodied as one or more software modules, e.g., computer implemented instructions configured to be stored in a non-transitory computer readable medium. Any suitable computer hardware can be utilized to operate the one or more software modules. Any suitable module(s) can be hosted commonly, or federated in any suitable manner. Any suitable module(s) can be integrated with each other or split in any suitable fashion.

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A telehealth system, comprising:
a telehealth conferencing module configured to allow a provider to communicate with a patient;
a health information module configured to provide patient medical record information to the provider to the telehealth conferencing module; and
a billing module operatively associated with the telehealth conferencing module and the health information module to receive service information relating to a telehealth visit and provide a billing output.

2. The system of claim 1, wherein the billing module is configured to aggregate the service information.

3. The system of claim 2, wherein the billing output includes a billing file having the service information.

4. The system of claim 3, wherein the billing file is a PDF file.

5. The system of claim 1, wherein the billing module is configured to determine one or more billing strategies for the provider.

6. The system of claim 4, wherein the billing output includes the one or more billing strategies.

7. The system of claim 5, wherein the one or more billing strategies includes a plurality of billing strategies.

8. The system of claim 7, wherein the billing module is configured to select a proposed billing strategy from the plurality of billing strategies.

9. The system of claim 8, wherein the billing module is configured to output the proposed billing strategy.

10. The system of claim 8, wherein the billing module is configured to determine one or more tasks based on substantive information in the service information and to look up a task value for each task from a task database.

11. The system of claim 10, wherein the billing module is configured to determine a billing time based on one or more of a manual time entry and/or a telehealth visit duration in the service information.

12. The system of claim 11, wherein the billing module is configured to provide a graphical user interface configured to receive a manually input time entry from a provider.

13. The system of claim 11, wherein the billing module is configured to select the proposed billing strategy based on a comparison of total billing time value versus total task value based on aggregated task values.

14. The system of claim 13, wherein the billing module is configured to provide the proposed billing strategy in a billing file.

15. The system of claim 14, wherein the billing module is configured to automatically formulate and output details relating to the telehealth visit using the proposed billing strategy.

16. The system of claim 1, wherein the telehealth conferencing module includes a videoconference pane and a health information pane, wherein the health information module is configured to output the patient medical record information to the health information pane.

17. The system of claim 16, wherein the health information pane includes an interface for a provider to indicate whether the patient medical record information is reviewed.

18. The system of claim 17, wherein the billing module is configured to confirm one or more billing tasks based on provider input to the health information pane.

19. A non-transitory computer readable medium comprising computer executable instructions configured to cause a computer to perform a method, the method comprising:
connecting a healthcare provider to a patient via a telehealth conferencing module;
receiving patient medical record information from an electronic medical record database;
displaying the patient medical record information in a health information pane;
receiving service information from the telehealth conferencing module;
receiving pre-teleconference time entry input and/or post-teleconference time entry input from the healthcare provider; and
determining a billing output based on the service information; and
outputting a billing file.

20. A telehealth system, comprising:
a health information module configured to provide patient medical record information to a telecommunication platform during a telehealth visit between a patient and a medical provider; and
a billing module operatively associated with the telecommunication platform and the health information module to receive service information relating to a telehealth visit and provide a billing output.
